(19) United States
(12) Patent Application Publication
(10) Pub. No.: US 2002/0016558 A1
(43) Pub. Date: Feb. 7, 2002

(54) Electrode arrangement for a shock wave source

(76) Inventors: Wilfried Haaks, Hamburg (DE); Horst-Hartwig Schwieker, Hamburg (DE)

Correspondence Address: Corporate Patent Counsel, U.S. Philips Corporation, 580 White Plains Road, Tarrytown, NY, 10591, US

(21) Appl. No.: 09/921,507

(22) Filed: Aug. 2, 2001

(30) Foreign Application Priority Data
Aug. 3, 2000 (DE) 10037790.4

(51) Int. Cl. A61B 17/225

(52) U.S. Cl. 601/4; 606/128

(57) ABSTRACT

The invention relates to an electrode arrangement for generating shock waves by electrical discharging between electrode tips (12, 13), which arrangement is particularly suitable for use as an ESWL (Extracorporal Shock Wave Lithotripsy) electrode in a shock wave source for the pulverization of kidney stones or as an electrode in an apparatus for Extracorporal Shock Wave Therapy (ESWT). The electrode arrangement is characterized notably in that at least one of the electrode tips (12, 13) can be replaceable arranged in an associated electrode holder (14, 15), the electrode tip and the electrode holder being provided with corresponding fitting contours so as to achieve mutual locking. This enables very economical replacement of spent electrode tips by the user.

The invention relates to an electrode arrangement for generating shock waves by electrical discharging between electrode tips which is particularly suitable as an ESWL (Extracorporal Shock Wave Lithotripsy) electrode in the shock wave source of a lithotripter for the pulverization of kidney stones or as an electrode in an apparatus for Extracorporal Shock Wave Therapy (ESWT).

Electrode arrangements of this kind are used, for example for underwater shock wave generation in lithotripsy where a spark gap is situated between the electrode tips in a first focal spot of an elliptical reflector whose second focal spot is aimed at the region in which a concrement, for example a kidney stone of a patient, is to be pulverized.

The electrode tips are subject to a comparatively high degree of consumption, so that the ESWL or ESWT electrode generally must be replaced after the treatment of from one to three patients. This is a drawback notably for economical reasons.

DE 38 14 468 discloses an electrode for the pulverization of kidney stones in which one of the electrode tips is soldered to a mandril which is inserted into a sleeve so as to be replaceable, the other electrode tip being secured, again by soldering, in a corresponding opening in a cage. The aim is to make the electrode re-usable by soldering in new electrode tips.

However, the foregoing approach has an essential drawback in that the electrode must be sent to the manufacturer for this purpose, so that even though the user achieves a small saving of costs in comparison with the purchase of a new electrode, the service costs are the same as in the case of electrodes that cannot be used again.

Therefore, it is a specific object of the invention to provide an electrode arrangement of the kind set forth which can be used at significantly lower costs and requires less maintenance work.

This object is achieved by means of an electrode arrangement of the kind set forth which is characterized in that at least one of the electrode tips can be replaceable arranged in an associated electrode holder, the electrode tip and the electrode holder being provided with corresponding fitting contours so as to achieve mutual locking.

An essential advantage of this solution consists in that the electrode tips can be simply replaced by the user himself This is not only more effective than the complete replacement of the electrode, but the costs are also significantly reduced.

The dependent claims relate to further advantageous embodiments of the invention.

The embodiment in conformity with claim 2 and its further elaboration in conformity with claim 3 offer the advantage that the electrode tip can be simply manufactured and that a known electrode holder need be structurally modified to a minor extent only.

However, when the embodiment disclosed in the claims 4 or 5 is chosen, no tensioning device will be required so that the mounting could be even simpler in some cases, be it that the manufacture could be slightly more complex.

Finally, the electrode arrangement in accordance with the invention is particularly suitable for use as a shock wave electrode or also as an ESWL electrode for use in a shock wave source for the pulverization of kidney stones or other concrements.

Further details, features and advantages of the invention will become apparent from the following description of a preferred embodiment as shown in the drawing.

Therein:

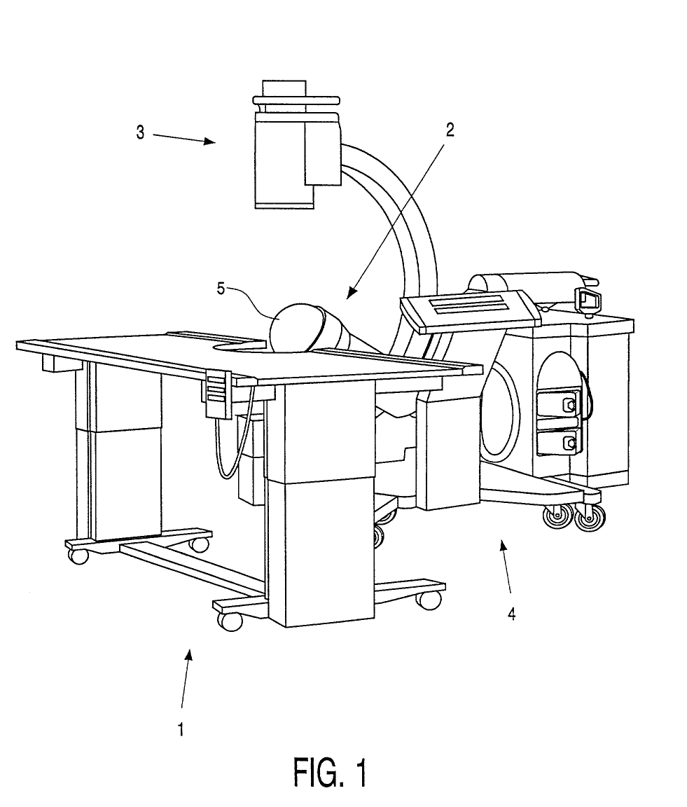

FIG. 1 is a diagrammatic view of a lithotripter;

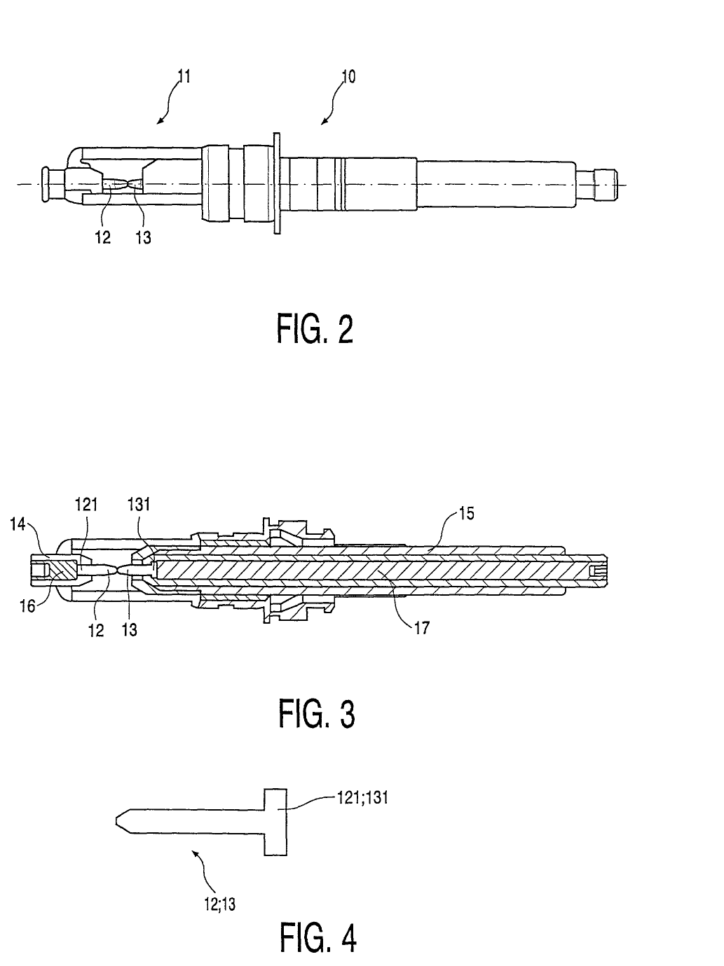

FIG. 2 is a diagrammatic view of an electrode arrangement;

FIG. 3 is a diagrammatic cross-sectional view of the electrode arrangement shown in FIG. 2, and

FIG. 4 is a view at an enlarged scale of an electrode tip.

A typical treatment station for lithotripsy, that is a lithotripter, comprises a patient table 1, a shock wave source 2, an X-ray apparatus 3 as well as various control units 4 as shown in FIG. 1. As is known, the shock wave source includes a rubber container 5 that is filled with water and is brought into contact with a patient, arranged on the patient table 1, in order to achieve an as smooth as possible transmission of the shock waves produced to the body of the patient.

The shock waves are produced by the electrode gap of an electrode arrangement (also referred to as shock wave electrode or ESWL electrode) which is situated within the rubber container, that is, in a first focal spot of an elliptical reflector. The known configuration and orientation are such that the second focal spot is situated within the body of the patient, that is, in a location where a kidney stone or other concrement is to be pulverized. The effectiveness of the treatment can be monitored by means of the X-ray apparatus 3.

FIG. 2 is an external view of a single electrode arrangement with a housing 10. The housing 10 includes known locking means for arranging the electrode in a shock wave source, as well as contacts for the supply of an electric voltage. At one side there is provided a cage 11 in which a first and a second electrode tip 12, 13 define a spark discharge gap.

FIG. 3 shows the internal construction of the above electrode arrangement and also the shape of the electrode tips 12, 13. At their ends which are remote from the spark discharge gap the electrode tips are provided with a respective pedestal-like peripheral extension which forms a respective projection 121, 131. The first electrode tip 12 is situated in a first electrode holder 14 whereas a second electrode holder 15 is provided for the second electrode tip 13. Both electrode holders are hollow and open at their (first) ends that are remote from the spark discharge gap, so that an electrode tip can be inserted in this first end and be fed therethrough as far as the second, opposite end of the electrode holders 14, 15.

At their second ends the electrode holders 14, 15 are provided with a respective bore having such a shape (inner contour) that the electrode tips 12, 13 can be fed through these bores so as to reach the spark discharge space as far as their projection 121, 131 that has a corresponding outer contour.

The corresponding fitting contours at the electrode tip and the second end of the electrode holder enable simple and accurate positioning of the free ends of the electrode tips in the spark discharge space.

The electrode tips are secured in position either in that the fitting of the contours is a press fit or, like in the case shown in FIG. 3, in that they are locked by a respective tensioning device in the form of a first and a second pressure screw 16, 17 which is introduced into the respective electrode holder in the axial direction and is screwed against the electrode tip by way of a thread provided on the inner wall of the electrode holder. At the same time an electrical connection is thus also established between the electrode tips 12, 13 and the relevant electrode holder 14 or 15, or an electrical terminal provided at that area.

Finally, FIG. 4 is an enlarged view of one of the electrode tips 12, 13 which illustrates notably the projection 121, 131.

When, after the treatment of from one to some three patients the electrode tips have been consumed so far that a spark discharge no longer occurs, the user loosens and removes the two pressure screws 16, 17 and removes the electrode tips from the electrode holders 14, 15. After insertion of new electrode tips 12, 13, the pressure screws are inserted and tightened again, thus enabling the operation to continue.

As an alternative for the described pressure screws, the electrode tips themselves may also be provided with a thread whereby they are screwed into a corresponding thread in the electrode holders so as to be locked therein.

A further possibility consists in providing a bayonet catch between the electrode tips and the electrode holders so as to achieve said locking.

In the embodiments described above the electrode tips are each time fed through the electrode holders from the inside so as to be locked. Conversely, of course, it is also possible to configure the locking in such a manner that the electrode tips are inserted into the electrode holders from the outside, that is, from the side of the spark discharge gap, and be secured thereto either by means of a thread or a bayonet catch.

1. An electrode arrangement for generating shock waves by electrical discharging between electrode tips, characterized in that at least one of the electrode tips (12, 13) can be replaceable arranged in an associated electrode holder (14, 15), the electrode tip and the electrode holder being provided with corresponding fitting contours so as to achieve mutual locking.

2. An electrode arrangement as claimed in claim 1, characterized in that the fitting contour is formed by at least one, essentially radially extending projection (121, 131) on the electrode tip as well as by a corresponding recess in the electrode holder (14, 15), the electrode tip (12, 13) with the projection being pressed there against by way of a tensioning device (16, 17).

3. An electrode arrangement as claimed in claim 2, characterized in that the tensioning device is formed by a pressure screw (16, 17) that can be screwed against the electrode tip (12, 13) in the electrode holder (14, 15) and at the same time establishes an electrical connection between the electrode tip and the electrode holder.

4. An electrode arrangement as claimed in claim 1, characterized in that the fitting contour is formed by corresponding threads on the electrode holder (14) and on the electrode tip (12, 13).

5. An electrode arrangement as claimed in claim 1, characterized in that the fitting contour is formed by a bayonet catch between the electrode holder (14, 15) and the electrode tip (12, 13).

6. A shock wave electrode (ESWL electrode) provided with an electrode arrangement as claimed in one of the preceding claims.

7. An electrode tip to be replaceable arranged in an electrode arrangement as claimed in one of the claims 1 to 5.

8. A shock wave source provided with an electrode arrangement as claimed in claim 1.

9. A lithotripter provided with a shock wave source as claimed in claim 8.
